(19) United States
(12) Patent Application Publication
(10) Pub. No.: US 2015/0351692 A1
(43) Pub. Date: Dec. 10, 2015

(54) ADJUSTABLE SEAT ASSEMBLY

(71) Applicant: Lear Corporation, Southfield, MI (US)

(72) Inventors: Michelle A. PERENY, Farmington Hills, MI (US); Samuel R. HANLON, Livonia, MI (US); Michael R. POWELL, Waterford, MI (US); Ashford Allen GALBREATH, Troy, MI (US); Richard L. HARBAUGH, South Lyon, MI (US); Terry O'BANNON, Royal Oak, MI (US); Gerald PATRICK, Shelby Township, MI (US); Karl HENN, New Hudson, MI (US)

(21) Appl. No.: 14/560,487

(22) Filed: Dec. 4, 2014

Related U.S. Application Data
(60) Provisional application No. 62/009,585, filed on Jun. 9, 2014.

(51) Int. Cl. A61B 5/00; A61B 5/103; B60N 2/22; A61B 5/11; B60N 2/44; B60N 2/00

(52) U.S. Cl. CPC A61B 5/6891; B60N 2/44; B60N 2/002; B60N 2/22; A61B 5/1116; A61B 5/1036

(57) ABSTRACT

A seat assembly is provided with a seat cushion, a seat back, and a head restraint. A plurality of sensors is operably connected to at least one of the seat cushion and the seat back to detect a seating position of an occupant. A media device is provided. A controller is in electrical communication with the plurality of sensors and the media device, and is configured to receive data from the plurality sensors, compare the data to determine if the occupant is seated evenly, and operate the media device to inform the occupant of an uneven posture seating position. A computer-program product is programmed for automatically displaying a pressure distribution upon a seat assembly. The displayed pressure distribution of the seat assembly is from measured pressure values from a plurality of sensors in a plurality of zones of the seat assembly.

### CROSS-REFERENCE TO RELATED APPLICATIONS

This application claims the benefit of U.S. provisional application Ser. No. 62/009,585 filed Jun. 9, 2014, the disclosure of which is hereby incorporated in its entirety by reference herein.

### TECHNICAL FIELD

Various embodiments relate to adjustable seat assemblies.

### BACKGROUND

An adjustable seat assembly is illustrated and described in U.S. Pat. No. 5,758,924, which issued on Jun. 2, 1998 to Lear Corporation.

### SUMMARY

According to at least one embodiment, a seat assembly is provided with a seat cushion. A seat back is adapted to be pivotally mounted adjacent the seat cushion. A plurality of sensors is operably connected to at least one of the seat cushion and the seat back to detect a seating position of an occupant. A media device is provided. A controller is in electrical communication with the plurality of sensors and the media device, and is configured to receive data from the plurality sensors, compare the data to determine if the occupant is seated evenly, and operate the media device to inform the occupant of an uneven posture seating position.

According to at least another embodiment, a computer-program product is embodied in a non-transitory computer readable medium that is programmed for automatically displaying a pressure distribution upon a seat assembly. The computer-program product includes instructions for receiving input indicative of measured pressure values from a plurality of sensors in a plurality of zones of a seat assembly. A range of the measured pressure values is determined. A range of colors is assigned to the range of the measured pressure values. Signals are provided to a display indicative of a color distribution to the plurality of zones of the seat assembly as a visual representation of pressure distribution upon the seat assembly.

According to at least another embodiment, a method for displaying pressure distribution of a seat assembly measures pressure values from a plurality of sensors in a plurality of zones of a seat assembly. A range of the measured pressure values is determined. A range of colors is assigned to the range of the measured pressure values. The seat assembly is displayed with the plurality of zones colored by the range of colors as a visual representation of pressure distribution upon the seat assembly.

### BRIEF DESCRIPTION OF THE DRAWINGS

FIG. 1 is a flowchart of a method for determining various adjustment positions of a vehicle seat assembly for various users, according to an embodiment;

FIG. 2 is a graph of data collected in the method of FIG. 1;

FIG. 3 is a front perspective view of a vehicle seat assembly, illustrated partially disassembled, according to an embodiment;

FIG. 4 is a display image for a vehicle seating system according to an embodiment;

FIG. 5 is another display image for the vehicle seating system of FIG. 4;

FIG. 6 is another display image for the vehicle seating system of FIG. 4;

FIG. 7 is another display image for the vehicle seating system of FIG. 4;

FIG. 8 is another display image for the vehicle seating system of FIG. 4;

FIG. 9 is another display image for the vehicle seating system of FIG. 4;

FIG. 10 is another display image for the vehicle seating system of FIG. 4;

FIG. 11 is another display image for the vehicle seating system of FIG. 4;

FIG. 12 is another display image for the vehicle seating system of FIG. 4;

FIG. 13 is a flowchart of a method for adjusting a vehicle seat assembly according to an embodiment; and

FIG. 14 is a flowchart of another method for adjusting a vehicle seat assembly according to another embodiment.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

A comfort, posture and wellness seating system for vehicle seat assemblies provides a visual interface with adjustment hardware organically or inorganically. The system may be employed to properly configure any new or existing seating system. The system can also address specific comfort, posture or preferences, such as thoracic support. The seating system objectifies comfort data and biomechanics knowledge to make the data transferable. The seating system connects biomechanical optimization to measurable engineering data to make a complete comfort, posture and wellness seating system.

The comfort, posture and wellness seating system integrates anthropometry, bio-mechanics, and historical seating comfort data. The seating system can be employed in original equipment for vehicles or in aftermarket products. Applicable markets include automotive, mass transit, airlines, etc., as well as non-vehicular seating such as office, home, commercial, and public venue seating.

Referring now to FIG. 1, a flowchart of a method for determining various adjustment positions of a vehicle seat assembly for various users is illustrated, according to an embodiment. Data collection may occur at block 20. The data collection 20 may include expert positioning of occupants for optimal comfort, posture and/or wellness by a doctor or chiropractor. The data collection 20 can be used at specific sites on an ongoing basis if required. The expert input provides a high level of expert comfort, posture and personalized fitting.

The data 22 may be based on anthropometry, body pressure distribution (BPD), status of actuators (such as pressure of inflatable air bladders, status of valves or the like), or other data that provides a comfort, posture and biomechanically optimized position of an adjustable vehicle seat assembly. The data is collected in a knowledge base 24 or table for setting adjustments based on categories of data. The knowledge base 24 may be compiled from the expert positioned data 20 and the occupant specific data 22. The setting adjustments from the knowledge base 24 are utilized for pre-set options 26 in a vehicle seat assembly 28. The setting adjustments 24 can be customized by a user at a controller or display 30.

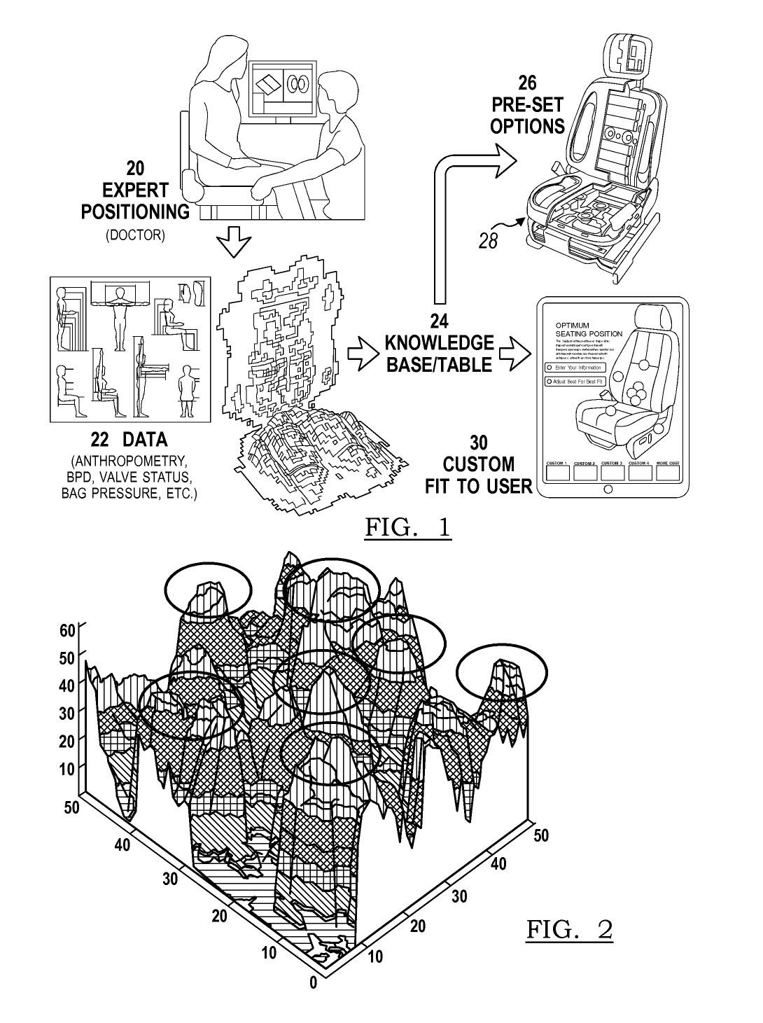

Referring to FIG. 2, input data 20, 22 can be plotted versus adjustment settings 20, 24 for high level categorization. The settings 20, 24 can be categorized by topology clustering for setting the pre-set options 26. Various setting options 26 may be provided for various types of driving. For example a touring setting may provide per package settings and basic comfort, posture and biomechanically optimized seating position recommendations. The touring setting may also provide optimal visibility, use of features and controls, and the like. A performance setting may be provided for active drivers to provide a more erect position with firmer seating. Additionally, a luxury setting may be more reclined with softer seating.

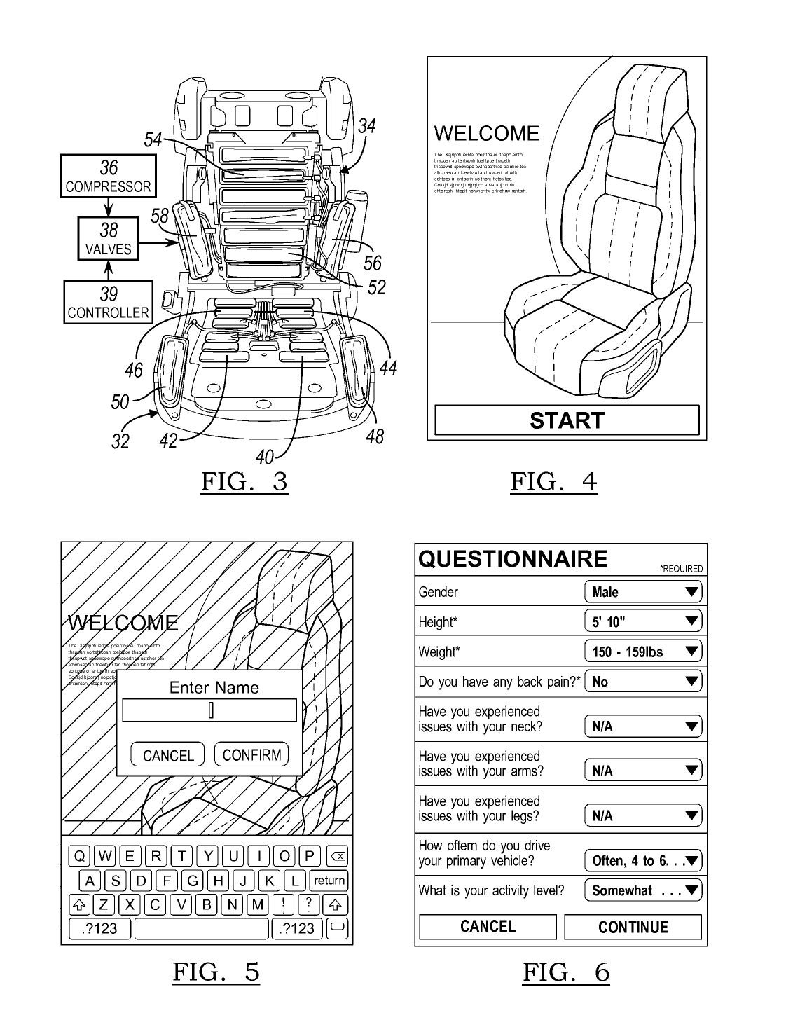

FIG. 3 illustrates the vehicle seat assembly 28 with a cover removed. The seat assembly 28 includes a seat cushion 32 adapted to be mounted for motor-driven adjustable translation in a fore and aft direction and in an up and down direction of a vehicle. The seat assembly 28 includes a seat back 34 pivotally connected to the seat cushion 32 to extend generally upright relative to the seat cushion 32 for motor-driven pivotal adjustment relative to the seat cushion 32. A head restraint (not shown) is mounted for motor-driven adjustable translation to the seat back 34.

At least one compressor 36 provides a source of air to the seat assembly 28. A plurality of valves 38 receive the compressed air and are controlled by a controller 39 for regulating compressed air into and out of the seat assembly 28. The seat cushion 32 includes a forward left air bladder 40, a forward right air bladder 42, a rear left air bladder 44, a rear right air bladder 46, a left side bolster air bladder 48, and a right side bolster air bladder 50. The seatback 34 includes a plurality of lumbar air bladders 52, a plurality of thoracic air bladders 54, a left side bolster air bladder 56, and a right side bolster air bladder 58. The valves 38 may be provided as a common valve bank that is housed in the seat back 34 or under the seat cushion 32; or the valves 38 may each be provided on each of the air bladders 40, 42, 44, 46, 48, 50, 52, 54, 56, 58. The compressor 36 may be provided in the seat back 34, the seat cushion 32 or concealed within the vehicle body. The controller 39 may be provided in a module under the seat cushion 32, and may be a multifunction controller that also controls other functions in the vehicle.

It is believed that supporting the thoracic region of the spine can reduce forces and support as much as one-third of the upper body mass. By increasing support of the upper body mass, loads are reduced on the muscles, ligaments, and spine and pelvic regions. Decreased load reduces fatigue on these areas of the body. The thoracic air bladders 54 are adjustable to provide the right degree of support in the correct location necessary to reduce such loading.

The controller 39 receives the adjustment settings 24 from pre-set data 26 or from customized data 30. The data may be input from an interface that is provided in the vehicle. The interface may be integrated into the vehicle, such as an instrument panel display that is in suitable wired or wireless communication with the controller 39. The interface may be remote, such as a personal digital assistant (PDA) including phones, tablets and the like. The interface may be provided as a smart phone application, wherein users enter relevant information about themselves. The smart phone interface may not require on-site expertise or seat properties. The remote interface permits a user to transport settings to each vehicle, such as personal passenger vehicles, airline seating, rental cars, and the like.

FIGS. 4-12 illustrate display images from an interface, such as a tablet. FIG. 4 illustrates a welcome screen wherein a data collection process is initiated. FIG. 5 illustrates a screen wherein a user name is input to identify the user. FIG. 6 illustrates another input screen wherein biometric and personal health data, such as wellness and activity level, is collected. This data is utilized to adjust the seat to the pre-set options 26, based on prior-collected data 22 in knowledge base 24.

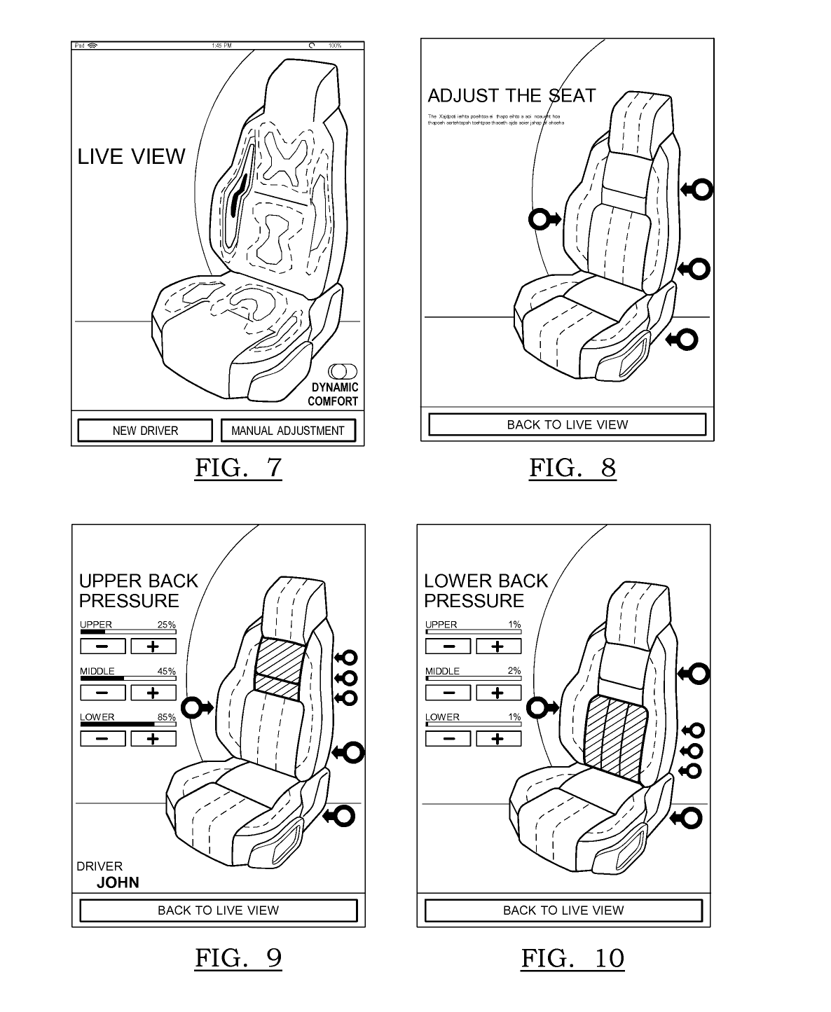

Each of the air bladders 40, 42, 44, 46, 48, 50, 52, 54, 56, 58 may include a pressure sensor to detect air pressure in the respective bladder 40, 42, 44, 46, 48, 50, 52, 54, 56, 58. Any pressure sensor is contemplated, such as a pneumatic pressure sensor at the outlet valve of each respective air bladder 40, 42, 44, 46, 48, 50, 52, 54, 56, 58. Pressure can also be sensed by contact pressure sensors disposed in front of or behind some or all of the respective air bladders, including on a front or rear surface thereof. The contact pressure sensors may include pressure-sensing mats, such as those available by Tekscan®, Inc. of 307 West First Street. South Boston, Mass. 02127-1309, USA. FIG. 7 illustrates a depiction of the vehicle seat assembly 28 with zones ranging in color to depict a distribution of pressure upon the seat. This visualization may assist an occupant in positioning upon the seat assembly 28 with live visual feedback. If manual adjustment is selected, FIG. 8 requests the occupant to select a zone of the seat for adjustment. Once a zone is selected, such as thoracic in FIG. 9 or lumbar in FIG. 10, incremental adjustment of each air bladder 52 by the occupant is permitted.

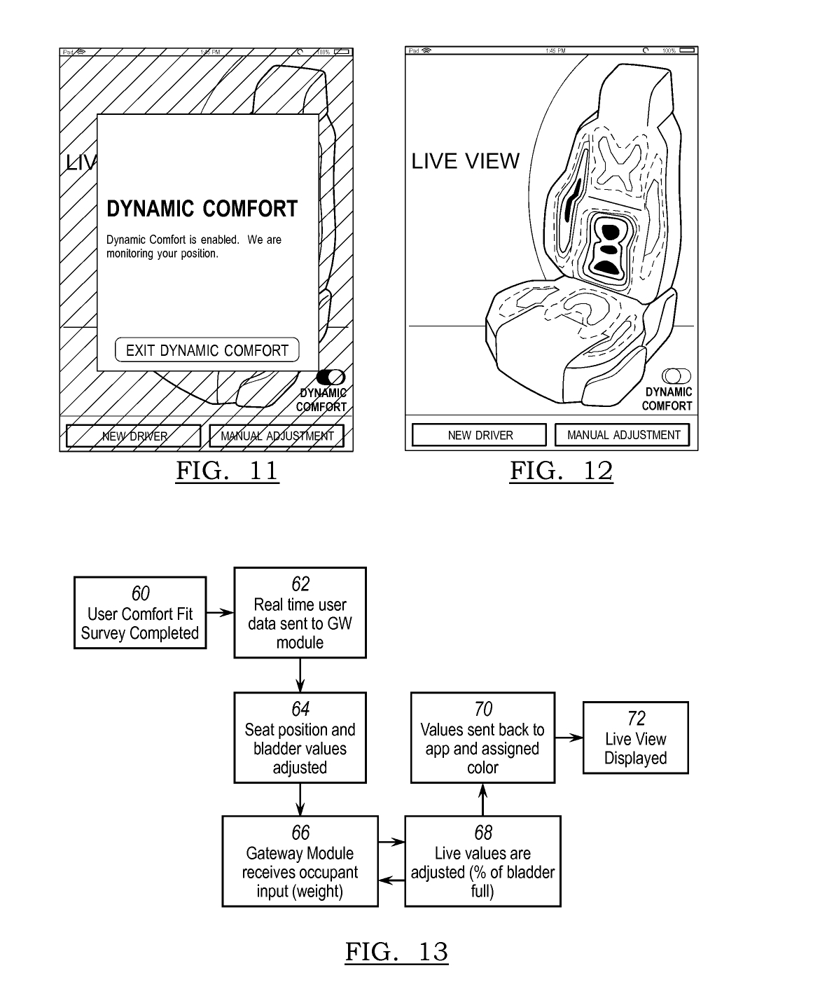

Referring again to FIG. 7, a dynamic comfort, posture and biomechanically optimized option is offered. Selection of the dynamic comfort option measures the pressure in the sensors at FIG. 11, and displays a live view as in FIG. 12. The controller 39 compares the sensor values, and if the controller 39 determines that the occupant is not seated evenly, the controller 39 balances the air pressure in opposing air bladders to balance the occupant seating position.

FIG. 13 depicts a flowchart for a method for adjusting the seat assembly 28 according to an embodiment. At block 60 the user comfort fit survey is completed. This process may be performed in an application on a PDA, tablet or smartphone, or in a controller in the vehicle. At block 62, real time user data is sent to a gateway module, or controller 39. In block 64, the seat assembly 28 is positioned, and the air bladder values are adjusted. At block 66, the gateway module receives an occupant weight distribution from the air bladder pressure sensors. At block 68 live values are adjusted, such as a percentage of bladder fullness. At block 70, the values are sent to the interface application, and the range of values is assigned a range of colors. In block 72, a live view is displayed, as in FIG. 12. Steps 66 and 68 are repeated for dynamically adjusting the seat assembly 28 to the occupant's position. Steps 66, 70 and 72 are repeated incrementally to provide the live view of the pressure distribution.

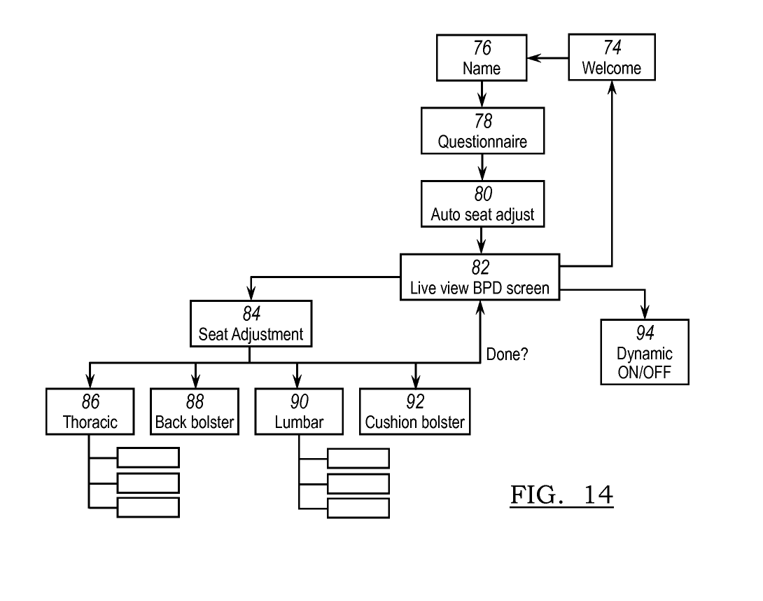

FIG. 14 depicts a flowchart for a method for adjusting the seat assembly 28 according to another embodiment. At block 74 a welcome screen initiates a data collection process. At block 76 a user name is input to identify the user. At block 78 another input screen includes a questionnaire wherein biometric and ergonomic data and activity level is collected. This data is utilized to adjust the seat at block 80 to the pre-set options 26, based on prior-collected data 22 in knowledge base 24. At block 82, a depiction of the vehicle seat assembly 28 is provided with zones ranging in color to depict a distribution of pressure upon the seat assembly 28. If manual adjustment is selected at block 84, the occupant can select a zone of the seat for adjustment, such as thoracic 86, back bolster 88, lumbar 90 and cushion bolster 92. Once manual adjustment is completed, the live view body pressure distribution (BPD) screen 82 is updated. If a dynamic mode is selected at block 84, then the adjustments and live view are updated based on the measurements from the pressure sensors and the biometric data.

While various embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A seat assembly comprising:
a seat cushion;
a seat back adapted to be pivotally mounted adjacent the seat cushion;
a plurality of sensors operably connected to at least one of the seat cushion and the seat back to detect a seating position of an occupant;
a media device; and
a controller in electrical communication with the plurality of sensors and the media device, configured to receive data from the plurality of sensors, compare the data to determine if the occupant is seated evenly, and operate the media device to inform the occupant of an uneven posture seating position.

2. The seat assembly of claim 1 wherein the plurality of sensors comprise:
at least one left side sensor; and
at least one right side sensor.

3. The seat assembly of claim 2 wherein the controller determines if the occupant is seated evenly left to right, and operates the media device to inform the occupant of an uneven left to right seating position.

4. The seat assembly of claim 1 wherein the media device comprises a display.

5. The seat assembly of claim 4 wherein the controller is configured to incrementally update the display to provide a visual feedback of adjustment of a seating position.

6. The seat assembly of claim 4 wherein the display comprises indicia indicative of the seat assembly with a plurality of zones corresponding to a plurality of zones of the seat assembly, each zone provided with one of the plurality of sensors.

7. The seat assembly of claim 6 wherein the zones are displayed ranging in color to represent a range of pressures measured by the plurality of sensors.

8. The seat assembly of claim 1 wherein the media device comprises an interactive user interface configured to receive input from the occupant.

9. The seat assembly of claim 8 wherein the media device is located remotely from the seat assembly.

10. The seat assembly of claim 9 wherein the media device is configured to communicate with the controller via wireless communication.

11. The seat assembly of claim 10 wherein the media device comprises a portable electronic device.

12. The seat assembly of claim 10 wherein the media device comprises at least one of a personal data assistant, a smartphone and a tablet.

13. A computer-program product embodied in a non-transitory computer readable medium that is programmed for automatically displaying a pressure distribution upon a seat assembly, the computer-program product comprising instructions for:
receiving input indicative of measured pressure values from a plurality of sensors in a plurality of zones of a seat assembly;
assigning a range of colors corresponding to a range of measured pressure values; and
providing signals to a display indicative of a color distribution to the plurality of zones of the seat assembly as a visual representation of pressure distribution upon the seat assembly.

14. The computer-program product of claim 13 further comprising instructions for repeating the instructions of claim 13 incrementally.

15. The computer-program product of claim 13 wherein the plurality of zones comprise a plurality of left and right zones of the seat assembly.

16. The computer-program product of claim 15 further comprising instructions for providing signals to the display indicative of an uneven pressure distribution across the plurality of left and right zones of the seat assembly.

17. A method for displaying pressure distribution of a seat assembly comprising steps of:
measuring pressure values from a plurality of sensors in a plurality of zones of a seat assembly;
determining a range of the measured pressure values;
assigning a range of colors to the range of the measured pressure values; and
displaying the seat assembly with the plurality of zones colored by the range of colors as a visual representation of pressure distribution upon the seat assembly.

18. A method comprising repeating the steps of claim 17 incrementally.

19. The method of claim 17 further comprising a step of measuring pressure values from a plurality of left and right sensors in a plurality of left and right zones of the seat assembly.

20. The method of claim 19 further comprising a step of displaying the seat assembly with the plurality of left and right zones colored by the range of colors.
